(19) United States
(12) Patent Application Publication
(10) Pub. No.: US 2009/0126737 A1
(43) Pub. Date: May 21, 2009

(54) OXYGEN SUPPLYING CIRCUIT FOR AN AIRCRAFT CREW MEMBER

(76) Inventors: Severine Aubonnet, Fontenay Le Fleury (FR); Andrew R.T Tatarek-Gintowt, Hants (GB)

Correspondence Address: JOHN S. PRATT, ESQ;KILPATRICK STOCKTON, LLP, 1100 PEACHTREE STREET, ATLANTA, GA, 30309, US

(21) Appl. No.: 12/093,002

(22) PCT Filed: Nov. 9, 2005
(86) PCT No.: PCT/EP05/13019

§ 371 Date: Jul. 15, 2008

(51) Int. Cl. A61M 16/06

(52) U.S. Cl. 128/204.29

(57) ABSTRACT

The invention relates to a breathing apparatus for an aircraft crew member, said apparatus comprising at least one breathing mask (620) provided with a demand regulator (810), said demand regulator being linked through a feedline to a central source of oxygen enriched air (600), so that oxygen enriched air is supplied to said breathing mask, and pressure regulating means (601,602,603, 604) provided on said feedline between said central source and said demand regulator and adapted, to level the pressure fluctuations of said oxygen enriched air supplied to said breathing mask.

This invention relates to breathing apparatus to deliver oxygen to an aircraft crew member, and more specifically to a breathing apparatus comprising a mask associated with a demand regulator.

A demand regulator is an essential feature to deliver oxygen or oxygen enriched air to an aircrew member through a mask in case of an emergency such as depressurization at high altitude and/or the occurrence of smoke in the cockpit. Such a device can also be worn as a preventive measure. A major, although non-exclusive application, lies in passenger airliners that can reach high altitudes, also called “jumbo” or “super-jumbo” aircraft of very large capacity.

At present, each airliner pilot has breathing apparatus comprising a mask fitted with a demand regulator connected to a source of breathing gas. Aviation regulations require that the mask can be put into place and supply oxygen to its wearer in less than 5 seconds. This result is generally achieved by using a mask provided with a demand regulator and a pneumatic harness that can be inflated and deflated, such as one of those described in documents FR-A-1 506 342, FR-A-2 784 900, EP-A-0 628 325, and U.S. Pat. Nos. 5,503,147, and 5,623,923. The source of gas under pressure must be capable of instantly delivering oxygen or air greatly enriched in oxygen at a pressure which is sufficient for inflating the harness and feeding the regulator of the mask. In general, the source is one or several cylinders of oxygen under pressure. The oxygen supply can be replaced by an on-board oxygen generator, such as a battery of on-board oxygen generator systems (OBOGS) fed with air derived from the compressor of one or more of the engines. OBOGS may also comprise a molecular sieve oxygen generating system (MSOGS) arranged to deliver oxygen-enriched air of desired oxygen concentration value by absorbing nitrogen from air fed to the system.

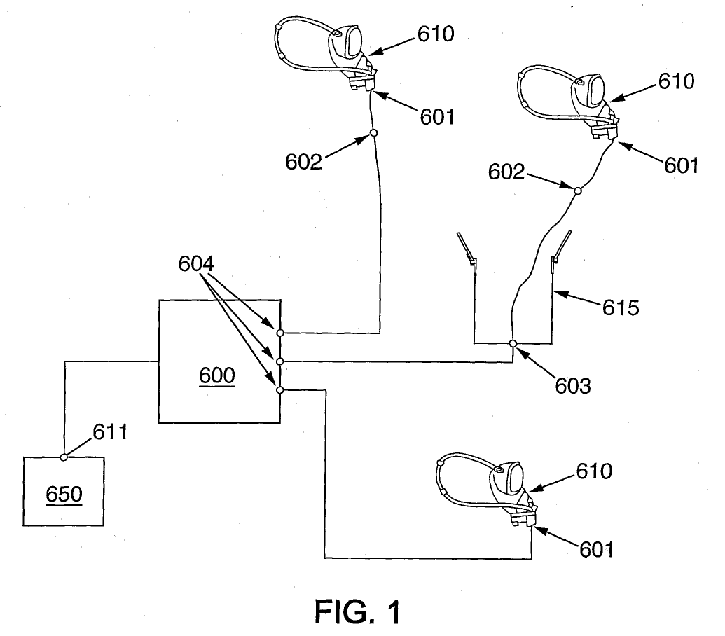

As seen in FIG. 1, the source of oxygen 610 is generally connected to a central system 600 from which a plurality of supply lines (or feedlines) starts. Among these lines, one can find the supply line to the crewmember masks 610 and/or the emergency masks for passengers (not shown in FIG. 1).

Whatever the source of oxygen may be, a demand regulator will experience significant fluctuations of the oxygen feed pressure, i.e. its operating pressure, fluctuations generated upstream the crewmember mask, and more specifically along the feedline between the central system and the mask. These pressure fluctuations can be characterized through their mean value and their amplitude as following:
the supply line mean value that is linked to the oxygen source, and may vary from one oxygen supplier to the other. Indeed the different oxygen suppliers for aircrafts on the market develop oxygen sources with a mean value function of their specificities and/or the aircraft manufacturer. This mean value generally ranges from 50 to 70 Psi,
the amplitude, or range, of the fluctuations that can result from either pressure fluctuations at the source outlet, or the friction losses along the feedlines. A pressure regulator may be provided at the inlet of the pressurized oxygen source with a pressure reducing device, such as a reducing valve. Nevertheless, these devices only dampens large pressure excursions: at the inlet of a demand regulator, fluctuations in the range of 45 Psi are commonly experienced. The largest amplitudes are noted for oxygen supplied from pressurized cylinders.

For a given oxygen supplier and the aircraft it is to be associated with, the mean value as well as the amplitude are well known.

The known demand regulators are entirely pneumatic devices that ensure the control of the oxygen content and of the instant flowrate of the air supplied to the mask wearer. Such control depends upon the cabin pressure altitude and the wearer's inspiration. The reliability of the demand regulator functions is directly linked to the stability of the supply line pressure.

To this day, only a fine and complex tuning of the demand regulator associated to the mask allow to deliver an adjustable, yet stable, oxygen or oxygen enriched air flowrate to the crewmember mask, besides the pressure fluctuations of the oxygen source.

The tuning difficulties are two-fold, as a manufactured demand regulator must be tuned to the oxygen source mean value and the pressure fluctuations of the supply line. As it would be too costly to develop a demand regulator specific to a supply line mean value, identical demand regulators have to be adjusted to different supply line mean values depending on the oxygen source they are associated to. That first tuning granted, a demand regulator requires additional adjustments to ensure its reliability through the entire range of the pressure fluctuations it is to be submitted to.

A brief illustration of the painstaking yet essential tuning procedure for a demand regulator will be described hereafter.

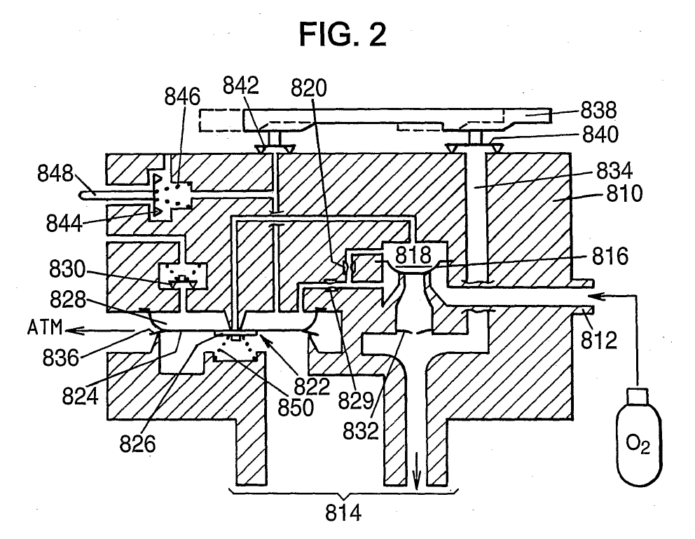

A known demand regulator is shown in FIG. 2. The demand regulator comprises a housing 810 made up of a plurality of assembled-together pieces, having an admission 812 for connection to a source of pressurized breathing gas, e.g. constituted by a cylinder of oxygen underpressure or a liquid oxygen converter. The housing also has a tube 814 for connection with the inside of a breathing mask (not shown) carrying regulator.

The housing 810 contains a pneumatic operated valve 816 constituted by a diaphragm co-operating with a fixed seat. A control chamber 818 defined by the rear surface of the main diaphragm and the housing is connected via a constriction 820 to the admission. When it is subjected to admission pressure, the diaphragm 816 is pressed against the seat, closing the passage in said seat, and separating the admission 812 from the tube 814.

The pressure that exists in the chamber 818 is controlled by a pilot valve 822. The pilot valve comprises a membrane 824 that is sensitive to pressure. The membrane 824 carries a shutter or closure member 826 which co-operates with a fixed seat to put the control chamber 818 into communication with a chamber 828 defined by the membrane 824, or to separate the chambers. A spring 850 sets the pressure threshold of membrane 824.

The chamber 828 also communicates with the admission via a constriction 829.

The pressure that exists inside the chamber 828 is limited by a valve 830 discharging to atmosphere and which prevents the high pressure in the chamber 828 exceeding a predetermined value.

To enable operation with dilution, an ejector 832 is interposed between the main valve 816 and the tube 814. When open, a passage 834 allows dilution air to arrive downstream from the ejector.

The pilot valve 822 is made in such a manner as to serve also as an exhaust valve. For this purpose, the membrane 824 has an annular rim 836 which bears against a seat for exhausting to the atmosphere.

The disposition described above is known and it is used by numerous demand regulators, so its operation need not be described in detail.

A demand regulator is generally manufactured without membrane 824 and pneumatic operated valve 816, as they are generally made of elastomer material that can be easily damaged. These two parts are assembled right before the tuning of the regulator.

As the regulator is to be tuned to a given mean value corresponding to an aircraft and its oxygen source, a complex tracking system is to be implemented to associate ahead of time the aircraft and its demand regulators. Knowing the oxygen source mean value, a battery of tests must be run to tune the demand regulator. Examples of the tests carried out are given hereafter.

After the membrane and the valve are assembled in the demand regulator body, a preliminary tuning is carried out at a common tuning pressure (50 Psi) for all demand regulators. The response time of the regulator (which corresponds to a comfort test, i.e. to ensure that the mask wearer can breath in effortlessly) is measured and adjusted to a desired value. This time corresponds to the breathing gas flow time from the valve to the wearer. The operating time of the membrane 824 is also tuned through in particular spring 850.

These two tunings among others are dependent upon the operating pressure. Additional tests, such as an endurance test including respiratory cycles subjected to the demand regulator, are also carried out to evaluate the aging, tuning and proper functioning of the valve and the membrane.

Finer adjustments are made with the actual operating pressure of the demand regulator, taking into account the whole range of fluctuations. The large pressure fluctuations that a demand regulator need to accommodate to make these finer adjustments all the more difficult, especially for the breathing comfort as vibrations can be observed at some operating pressures.

In some operating mode of a demand regulator, air is mixed to the oxygen supplied by the oxygen source. Air may be supplied to the regulator by means of an ejector (832 on FIG. 2) that is to be sized up. The ejector characteristics are based on the optimization busing a Computorized Fluid Dynamics (CDF) software) of a known regulator, taking into account variables such as altitude, temperature and the theoretical pressure upstream the ejector. Therefore, the operating pressure of the regulator also impacts the ejector characteristics.

A direct consequence of this fine tuning is a high and costly rejection rate of the demand regulators after manufacturing. If a demand regulator fails one of these tests, and the operator cannot adjust the valve or the membrane spring, the regulator will be discarded. The high rejection rate is mainly due to the high constraints on each demand regulator as one regulator is to perform within normal parameters at the 50 Psi test value, at oxygen supplier mean value, and along the fluctuation ranges.

It would therefore be highly desirable to develop a demand regulator that does not require so much extensive tuning, and that displays a reduced rejection rate. It would also be desirable to develop a demand regulator that is less sensitive to the multiplicity of pressure mean values and fluctuations, and lessen their impact on the regulator performances.

Accordingly, the present invention provides a pressure regulator according to claim 1.

The applicant took advantage of the observation that whatever the mean value and the amplitude of the pressure fluctuations may be at the inlet of the demand regulator, the performances of the regulator after proper tuning are equivalent. In other words, the same flow of oxygen is delivered to the mask wearer, independently of the oxygen source, provided the demand regulator is properly tuned. It is actually the goal of the whole tuning process to render the regulator operations independent of the supplied oxygen characteristics.

Therefore, the applicant thought of interposing pressure regulating means between the central system and the demand regulator along the feedline in order to lower the mean value of the oxygen pressure fluctuations to a determined value, and reduce the fluctuations amplitude within a controlled range at the same time. Such pressure regulating means lessen the impact of the multiple oxygen sources. Regardless of the oxygen source and the feed lines upstream the mask, a demand regulator according to the invention experiences an operating pressure of known and controlled variations.

Thus tests only need to be carried out for all demand regulators at that determined pressure value, and taking into account the controlled range of fluctuations. Number of test is considerably reduced, whatever the oxygen supplier the demand regulator will be associated with. As the constraints on the demand regulator are limited, the rejection rate is considerably lowered. Furthermore, within the well known fluctuation range around the determined mean value, the performances of the regulator are not altered.

Other features and advantages of this invention will further appear in the hereafter description when considered in connection to the accompanying drawings, wherein:

FIG. 1 illustrates a principle diagram of the oxygen supply source and distribution system on board an aircraft;

FIG. 2 illustrates a sectional view of a known demand regulator;

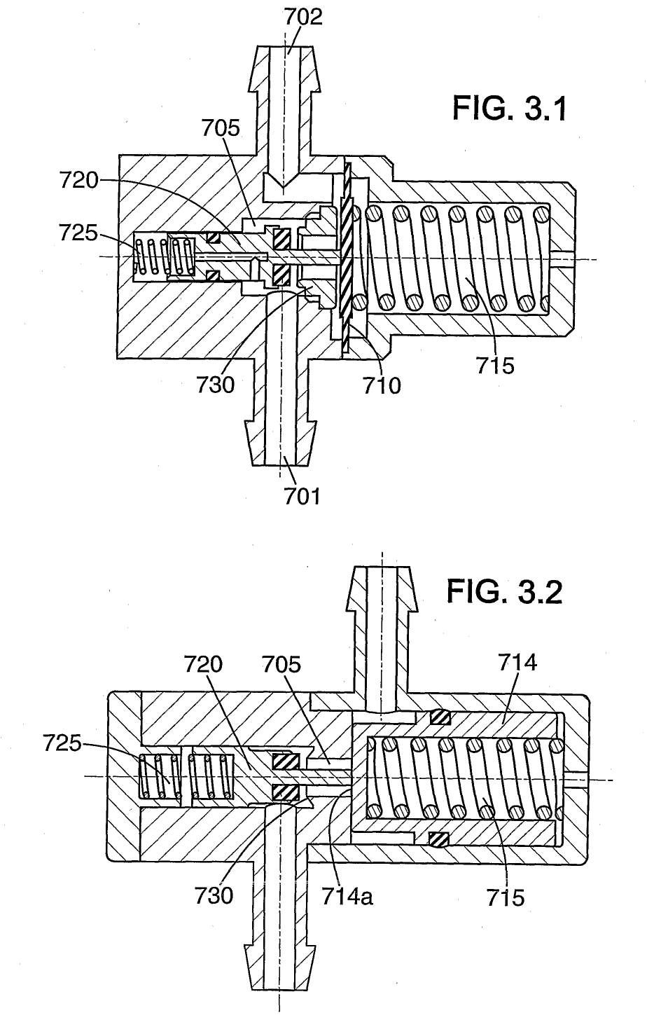

FIG. 3.1 illustrates a sectional view of a pressure reducing means with a flexible membrane;

FIG. 3.2 illustrates a sectional view of a pressure reducing means with a hollow body comprising the biasing means;

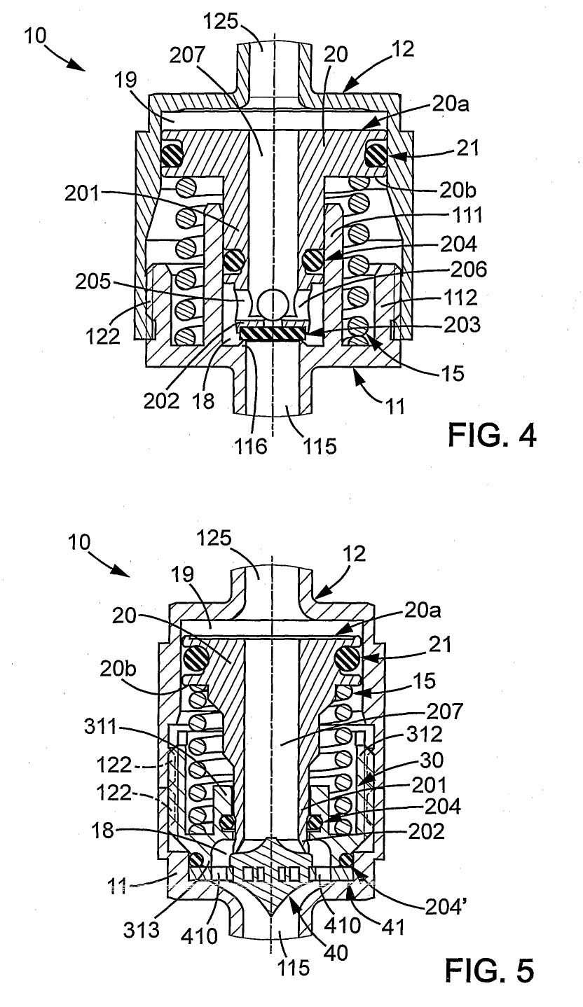

FIG. 4 illustrates a sectional view of a first embodiment of the pressure regulating means according to the present invention; and,

FIG. 3 illustrates a sectional view of a second embodiment of the pressure regulating means according to the present invention.

The pressure reducing means, which acts as a pressure regulator that delivers a regulated lowered pressure, as well as a pressure fluctuation limiter, may take the form of a pressure reducing device. It may be located at different locations along the supply line (or feed line) of the aircraft, between the central system and the mask of the crewmember. Four main locations can be defined.

A first location 601 as seen on FIG. 1 corresponds to the pressure reducing means directly provided at the inlet of the demand regulator of mask 610. Such a pressure reducing device being of small dimensions and light in weight, it is well suited to be positioned in this first location 601. The device may be of a cylinder form to be fit into the demand regulator inlet.

When storage box 615, connected to the oxygen feedline, is available for the mask 610, a convenient second location 602 is along the feedline between the storage box and the demand regulator. A pressure reducing device of cylinder form can be provided locally along the feedline. The storage box is preferably located close to the crewmember. That same location is applicable in the absence of the storage box, i.e. along the feedline between central system 600 and mask 610.

A third example of location is at the inlet 603 of the oxygen feedline into the storage box 615. A convenient form of the pressure reducing device may be a parallelepipedal form to easily attach the device to the box.

A fourth example of location, when a storage box may not be available, is along the feedline next to, or within, the feedline adapter (connecting means) 604 onto the central system.

Pressure regulating devices are known from U.S. Pat. Nos. 3,437,109 and 4,226,257. Such devices generally comprise:
an inlet or inlet end portion,
an outlet or outlet end portion,
a regulation chamber, or pressure chamber, interposed between the inlet and the outlet, so that the inlet is in fluid communication with the outlet through the regulation chamber, the regulation chamber providing a substantially constant pressure to the outlet,
piston means adapted to move at least partially within the regulation chamber between a first position wherein the piston means seals the fluid communication between the inlet and the outlet and a second position wherein oxygen enriched air can flow from the inlet to the outlet,
biasing means adapted to bias the piston means towards the second position, and
urging means adapted to counter the biasing of said piston means thanks to a force function of the regulation chamber pressure.

The urging means may comprise a flexible membrane 710, as seen in FIG. 3.1, which faces through its first side the regulation chamber 705. The biasing means 715 is actually biasing the membrane on its second side. In the regulation chamber 705, the piston means 720 is maintained in contact with the first side of the membrane through a spring 725 that urges said piston means 720 towards the membrane 710. The regulation is obtained through the sum of the forces applied to the membrane.

Starting from the second position of the piston means 720, as the pressure increases within the regulation chamber 705, the pressure applied to the membrane increases, said membrane 710 deflects and compresses the biasing means 715. The piston means 720 follows the membrane 710 displacement thanks to the spring 725. When the piston means 720 reached the first position, as shown in FIG. 3.1, it seals the fluid communication between the inlet 701 and the outlet 702. The outlet pressure then decreases, which lowers the pressure force applied to the membrane 710. As the biasing force from the biasing means 715 on its second side becomes higher than the pressure force applied on its first side, the biasing means 715 biases 710 the piston means 720 back to the second position through the flexible membrane 720. The fluid communication is reopened.

Another embodiment of the pressure regulation means consists in substituting the flexible membrane 710 with a biasing body 714, preferably hollow. Biasing body 714 faces the regulation chamber 705 and is biased through biasing means 715 to push the piston means 720 to its second position. Piston means 720 is also maintained in contact with biasing body 714 through a spring 725 as in the example of FIG. 3.1. Biasing body 714 further faces the regulation chamber 705 through a solid, preferably flat, surface 714a so that as the pressure in said regulation chamber increases, biasing body 714 is moved away while compressing biasing means 715. The regulation mechanism is similar to the one described for the embodiment with a flexible membrane.

In both instances, the first position corresponds to the piston means in contact with valve seat 730 on FIG. 3.1 and FIG. 3.2 which ensures the sealing of the fluid communication from the inlet to the outlet of the pressure regulating means.

Other embodiments will be described in details here after. A first embodiment of the regulating device according to the present invention is shown on FIG. 4. The pressure regulating device 10, or pressure regulator, comprises a valve actuator housing 12 and a main body 11 screwed to each other through threaded section 122 on an outer projecting generally annular extension 112 of said main body 11. Housing 12 may further be sealed with respect to the main body 11 of the regulator by use of a sealing gasket (not shown in FIG. 1). Housing 12 and main body 11 form a regulator body.

The regulator 10 further comprises an inlet or inlet end portion 115 provided on the main body 11 and an outlet or outlet end portion 125 provided on the valve actuator housing 12. The inlet end portion 115 is adapted for connection to the feedline coming from the central source of oxygen. The inlet end portion 115 includes an inlet passage which leads to a pressure chamber, or regulation chamber 18 defined within regulator main body 11. In communication with pressure chamber 18 therewith is an outlet passage disposed in the outlet end portion 125 of the valve actuator housing 12.

The valve actuator housing 12, in the form of a hollow bonnet, is disposed adjacent to pressure chamber 18. A floating piston 20 is slidably mounted within housing 12 for movement therein in a sealed manner by means of a sealing ring 21 which is disposed in the outer circumferential surface of piston 20 so as to interact with the interior wall of housing 12. Piston 20 has associated therewith a valve stem 201 and a valve member 202. The valve member 202 and valve stem 201, being integrally formed with piston 20, undergo movement therewith. Valve member 202 is adapted to engage a valve seat 116 formed as part of the inlet end portion 115. In this regard, the valve member 202 carries a sealing element 203. The valve stem 201 carries a sealing member 204 which sealingly engages an inner projecting generally annular extension 111 of the main body 11 in which the valve stem 201 is mounted. The floating piston 20 and thus the valve stem 201 are adapted to snugly fit into inner extension 111 for moving forward and away from valve seat 116.

The valve member 202 comprises connecting passages 205 and 206, while valve stem 201 comprises connecting passage 207 whereby pressure in chamber 18 is openly communicated to the outlet chamber 19 defined in the interior of housing 12 and end surface 20a of piston 20. Outlet chamber 19 is in direct communication with the outlet end portion 125.

A biasing means in the form of coil spring 15 is provided within the valve actuator housing 12, partly between the inner and outer extensions 111 and 112 to engage the opposite end surface 20b of piston 20 so as to urge the latter, as well as the valve stem 201 and valve member 202, away from valve seat 116. Coil spring 15 engages on its opposite side the inner surface of main body 11 from which extensions 111 and 112 are projected.

In the general use of the regulator 10, a fluctuating supply pressure may be supplied through inlet end portion 115. The principal purpose of regulator 10, therefore, is to provide or rather maintain a constant output pressure, which is communicated, via the rest of the feedline to the demand regulator.

Accordingly, a regulated pressure is to be maintained in the pressure chamber 18 which in turn communicates such regulated pressure to the outlet chamber 19, and consequently to the outlet passage disposed in the outlet end portion 125. Furthermore, the regulated pressure in chamber 18 is communicated to the end surface 20a of piston 20 and outer chamber 19 by the passages 205, 206 and 207 respectively. The regulated pressure acting on surface 20a of piston 20 imparts a specified force on the piston 20 tending to urge the same away from the outlet end portion 125 so as to close valve member 202 against the valve seat 116. Surface 20a corresponds to the urging means described here before. To counteract this tendency, the biasing means, such as spring 15, engage surface 20b of the piston so as to be operable to provide substantially a constant force on piston 20 throughout its range of travel with respect to the regulated pressure in chamber 18. The force of spring 15 is transmitted to the piston through end surface 20b.

Should the valve member 202 tend to close against valve seat 116 so that the pressure in chamber 18 falls below the desired regulated level, the force applied to end surface 20a of the piston proportionately decreases and the biasing means 15 would tend to open the valve member 202 so as to increase the pressure in chamber 18 and corresponding pressure applied to surface 20a of the piston. Conversely, if the valve member 202 tends to move away from valve seat 116, the pressure in chamber 18 would exceed the desired regulated level and the corresponding greater amount of force applied to end surface 20a of the piston would tend to move the latter against the biasing means force, so as to close down the valve member 202 until the regulated pressure level is reached. Thanks to counterbalancing forces, a substantially regulated pressure level is maintained at the outlet end portion 125.

A second embodiment of the regulating device according to the present invention is shown on FIG. 5. The same reference numbers refer to the same elements unless mentioned otherwise. Furthermore, only the dissimilarities between the two embodiments will be highlighted.

The pressure regulating device 10, or pressure regulator, comprises a valve actuator housing 12 and a main body 11 screwed to each other through threaded section 122 of a seal retainer 30 hosted within housing 12 and main body 11. Threaded section is carried on an outer projecting generally annular extension 312 of said seal retainer 30.

The regulator 10 further comprises an inlet end portion 115 provided on the main body 11 and an outlet end portion 125 provided on the valve actuator housing 12. The inlet end portion 115 includes an inlet passage which is in direct communication with a pressure chamber 18 defined between main body 11 and seal retainer 30. Seal retainer 30 is a hollow element which further comprises an inner projecting generally annular extension 311 in which valve stem 201 is mounted. On the opposite side of annular extensions 311 and 312, seal retainer 30 is prolonged with another annular extension 313 that defines with the inside of main body 11 said pressure chamber 18. A generally flat seal disk 41 is squeezed between annular extension 313 and main body 11 inner walls. Seal disk 41 separates pressure chamber 18 into two subchambers, one in direct communication with inlet passage within inlet end portion 115, and the other one in communication with connecting passage 207 of piston 20.

A sealing element 40 is provided on sealing disk 41. Sealing element is preferably of a generally cylindrical form, and localized on sealing disk 41 opposite valve member 202 of piston 20. Openings 410 are provided on sealing disk 41 to allow flow communication between the subchambers. Through openings 410 and connecting passage 207, pressure is communicated from inlet end portion 115 to outlet chamber 19 and outlet end portion 125. The floating piston 20 and thus the valve stem 201 are adapted to snugly fit into inner extension 311 for moving forward and away from sealing element 40. Furthermore, valve stem 202 is prolonged as an annular element adapted to engage sealing element 40 when urged towards the later by pressure force on the end surface 20a of piston 20, thus closing communication between inlet 115 and outlet 125 end portions. Biasing means 15 are provided between seal retainer 30 (between inner and outer extensions 311 and 312), and opposite end surface 20b of piston 20.

A sealing member 204′ can be provided to seal disk 41 between annular extension 313 and main body 11. Another seal member 204 can be provided between seal retainer 30 and valve stem 201.

Functioning of this embodiment is similar to the functioning described for the first embodiment of FIG. 4, as sealing element 40 acts as a valve seat. Thanks to counterbalancing forces, a substantially regulated pressure level is maintained at the outlet end portion 125.

1. A breathing apparatus for an aircraft crew member, said apparatus comprising:
at least one breathing mask provided with a demand regulator, said demand regulator being linked through a feedline to a central source of oxygen enriched air, so that oxygen enriched air is supplied to said breathing mask, said oxygen enriched air presenting pressure fluctuations characterized by a mean value and a fluctuation amplitude around said mean value, and
pressure regulating means provided on said feedline between said central source and said demand regulator and adapted to lower said mean value to a determined value, and level said fluctuation amplitude within a controlled range of pressure fluctuations.

2. A breathing apparatus according to claim 1, wherein said pressure regulation means comprises:
an inlet,
an outlet,
a regulation chamber interposed between said inlet and said outlet, so that said inlet is in fluid communication with said outlet through said regulation chamber, said regulation chamber providing a substantially constant pressure to said outlet,
piston means adapted to move at least partially within said regulation chamber between a first position wherein said piston means seals the fluid communication between said inlet and said outlet and a second position wherein oxygen enriched air can flow from said inlet to said outlet,
biasing means adapted to bias said piston means towards said second position, and
urging means adapted to counter the biasing of said piston means thanks to a force function of the regulation chamber pressure.

3. A breathing apparatus according to the previous claims claim 1, wherein the urging means comprises a membrane subjected to the regulation chamber pressure.

4. A breathing apparatus according to claim 1, wherein the urging means comprises an urging body with a solid surface subjected to the regulation chamber pressure.

5. A breathing apparatus according to claim 1, wherein the pressure regulating means comprises a valve actuator housing 2 disposed adjacent to said regulation chamber, said piston means being mounted in a sealed manner in said valve actuator housing, said piston means including a valve stem and valve member for movement therewith, said valve stem being slidably disposed in said regulation chamber and said inlet having a valve seat adjacent to said regulation chamber and adapted to cooperate with said valve member to close the communication between said inlet and said outlet, a connecting passage provided within said piston means for communicating pressure in said regulation chamber to an end surface of said piston means so that the latter tends to urge said valve member against said valve seat, the biasing means being located in said valve actuator housing for biasing said piston means so that said valve member tends to move away from said valve seat.

6. A breathing apparatus according to claim 1, wherein the demand regulator comprises an inlet and the pressure regulating means are provided at said inlet.

7. A breathing apparatus according to claim 1, wherein the feedline is connected to the central source through connecting means, the pressure regulating means being provided next to or within said connecting means.

8. A breathing apparatus according to claim 1, further comprising a storage box for storing the mask, said storage box being connected to the feedline through an inlet, and the pressure regulating means being provided at said inlet.

9. A breathing apparatus according to claim 1, further comprising a storage box for storing the mask, said storage box being connected to the feedline, and the pressure regulating means being provided between said storage box and the demand regulator.
